SEQUENCE LISTING (24 sequences)

SEQ ID NO: 1 - DNA, 20 bp, Artificial Sequence
  Human Fibroblast Growth Factor Receptor-1 forward primer
ggcccagacaacctgcctta

SEQ ID NO: 2 - DNA, 26 bp, Artificial Sequence
  Human Fibroblast Growth Factor Receptor-1 probe
ccaccgacaaagagatggaggtgctt

SEQ ID NO: 3 - DNA, 20 bp, Artificial Sequence
  Human Fibroblast Growth Factor Receptor-1 reverse primer
tgcgtcctcaaaggagacat

SEQ ID NO: 4 - DNA, 20 bp, Artificial Sequence
  Human Fibroblast Growth Factor Receptor-2 forward primer
gctgctgaaggaaggacaca

SEQ ID NO: 5 - DNA, 23 bp, Artificial Sequence
  Human Fibroblast Growth Factor Receptor-2 probe
agccagccaactgcaccaacgaa

SEQ ID NO: 6 - DNA, 19 bp, Artificial Sequence
  Human Fibroblast Growth Factor Receptor-2 reverse primer
gcatgccaacagtccctca

SEQ ID NO: 7 - DNA, 19 bp, Artificial Sequence
  Human Fibroblast Growth Factor Receptor-3 forward primer
ctcgggagatgacgaagac

SEQ ID NO: 8 - DNA, 23 bp, Artificial Sequence
  Human Fibroblast Growth Factor Receptor-3 probe
agccagccaactgcaccaacgaa

SEQ ID NO: 9 - DNA, 17 bp, Artificial Sequence
  Human Fibroblast Growth Factor Receptor-3 reverse primer
cgggccgtgtccagtaa

SEQ ID NO: 10 - DNA, 20 bp, Artificial Sequence
  Human cytosolic beta-Actin forward primer
tccaccttccagcagatgtg

SEQ ID NO: 11 - DNA, 28 bp, Artificial Sequence
  Human cytosolic beta-Actin probe
atcagcaagcaggagtatgacgagtccg

SEQ ID NO: 12 - DNA, 21 bp, Artificial Sequence
  Human cytosolic beta-Actin reverse primer
ctagaagcatttgcggtggac

SEQ ID NO: 13 - DNA, 19 bp, Artificial Sequence
  Human beta-2 microglobulin forward primer
gtctcgctccgtggcctta

SEQ ID NO: 14 - DNA, 26 bp, Artificial Sequence
  Human beta-2 microglobulin probe
tgctcgcgctactctctctttctggc

SEQ ID NO: 15 - DNA, 21 bp, Artificial Sequence
  Human beta-2 microglobulin reverse primer
tggagtacgctggatagcctc

SEQ ID NO: 16 - DNA, 20 bp, Artificial Sequence
  Human L32 ribosomal protein forward primer
ctggtccacaacgtcaagga

SEQ ID NO: 17 - DNA, 22 bp, Artificial Sequence
  Human L32 ribosomal protein probe
tggaagtgctgctgatgtgcaa

SEQ ID NO: 18 - DNA, 21 bp, Artificial Sequence
  Human L32 ribosomal protein reverse primer
agcgatctcggcacagtaaga

SEQ ID NO: 19 - DNA, 20 bp, Artificial Sequence
  Human glyceraldehyde 3-phosphate dehydrogenase forward primer
ctgggctacactgagcacca

SEQ ID NO: 20 - DNA, 28 bp, Artificial Sequence
  Human glyceraldehyde 3-phosphate dehydrogenase probe
tggtctcctctgacttcaacagcgacac

SEQ ID NO: 21 - DNA, 20 bp, Artificial Sequence
  Human glyceraldehyde 3-phosphate dehydrogenase reverse primer
cagcgtcaaaggtggaggag

SEQ ID NO: 22 - DNA, 5917 bp, Artificial Sequence
  NM_023110.2 (FGFR1)
agatgcaggggcgcaaacgccaaaggagaccaggctgtaggaagagaagggcagagcgccggacagctcggcccgctccccgtcctttggggccgcggctggggaactacaaggcccagcaggcagctgcagggggcggaggcggaggagggaccagcgcgggtgggagtgagagagcgagccctcgcgccccgccggcgcatagcgctcggagcgctcttgcggccacaggcgcggcgtcctcggcggcgggcggcagctagcgggagccgggacgccggtgcagccgcagcgcgcggaggaacccgggtgtgccgggagctgggcggccacgtccggacgggaccgagacccctcgtagcgcattgcggcgacctcgccttccccggccgcgagcgcgccgctgcttgaaaagccgcggaacccaaggacttttctccggtccgagctcggggcgccccgcagggcgcacggtacccgtgctgcagtcgggcacgccgcggcgccggggcctccgcagggcgatggagcccggtctgcaaggaaagtgaggcgccgccgctgcgttctggaggaggggggcacaaggtctggagaccccgggtggcggacgggagccctccccccgccccgcctccggggcaccagctccggctccattgttcccgcccgggctggaggcgccgagcaccgagcgccgccgggagtcgagcgccggccgcggagctcttgcgaccccgccaggacccgaacagagcccgggggcggcgggccggagccggggacgcgggcacacgcccgctcgcacaagccacggcggactctcccgaggcggaacctccacgccgagcgagggtcagtttgaaaaggaggatcgagctcactgtggagtatccatggagatgtggagccttgtcaccaacctctaactgcagaactgggatgtggagctggaagtgcctcctcttctgggctgtgctggtcacagccacactctgcaccgctaggccgtccccgaccttgcctgaacaagcccagccctggggagcccctgtggaagtggagtccttcctggtccaccccggtgacctgctgcagcttcgctgtcggctgcgggacgatgtgcagagcatcaactggctgcgggacggggtgcagctggcggaaagcaaccgcacccgcatcacaggggaggaggtggaggtgcaggactccgtgcccgcagactccggcctctatgcttgcgtaaccagcagcccctcgggcagtgacaccacctacttctccgtcaatgtttcagatgctctcccctcctcggaggatgatgatgatgatgatgactcctcttcagaggagaaagaaacagataacaccaaaccaaaccgtatgcccgtagctccatattggacatccccagaaaagatggaaaagaaattgcatgcagtgccggctgccaagacagtgaagttcaaatgcccttccagtgggaccccaaaccccacactgcgctggttgaaaaatggcaaagaattcaaacctgaccacagaattggaggctacaaggtccgttatgccacctggagcatcataatggactctgtggtgccctctgacaagggcaactacacctgcattgtggagaatgagtacggcagcatcaaccacacataccagctggatgtcgtggagcggtcccctcaccggcccatcctgcaagcagggttgcccgccaacaaaacagtggccctgggtagcaacgtggagttcatgtgtaaggtgtacagtgacccgcagccgcacatccagtggctaaagcacatcgaggtgaatgggagcaagattggcccagacaacctgccttatgtccagatcttgaagactgctggagttaataccaccgacaaagagatggaggtgcttcacttaagaaatgtctcctttgaggacgcaggggagtatacgtgcttggcgggtaactctatcggactctcccatcactctgcatggttgaccgttctggaagccctggaagagaggccggcagtgatgacctcgcccctgtacctggagatcatcatctattgcacaggggccttcctcatctcctgcatggtggggtcggtcatcgtctacaagatgaagagtggtaccaagaagagtgacttccacagccagatggctgtgcacaagctggccaagagcatccctctgcgcagacaggtaacagtgtctgctgactccagtgcatccatgaactctggggttcttctggttcggccatcacggctctcctccagtgggactcccatgctagcaggggtctctgagtatgagcttcccgaagaccctcgctgggagctgcctcgggacagactggtcttaggcaaacccctgggagagggctgctttgggcaggtggtgttggcagaggctatcgggctggacaaggacaaacccaaccgtgtgaccaaagtggctgtgaagatgttgaagtcggacgcaacagagaaagacttgtcagacctgatctcagaaatggagatgatgaagatgatcgggaagcataagaatatcatcaacctgctgggggcctgcacgcaggatggtcccttgtatgtcatcgtggagtatgcctccaagggcaacctgcgggagtacctgcaggcccggaggcccccagggctggaatactgctacaaccccagccacaacccagaggagcagctctcctccaaggacctggtgtcctgcgcctaccaggtggcccgaggcatggagtatctggcctccaagaagtgcatacaccgagacctggcagccaggaatgtcctggtgacagaggacaatgtgatgaagatagcagactttggcctcgcacgggacattcaccacatcgactactataaaaagacaaccaacggccgactgcctgtgaagtggatggcacccgaggcattatttgaccggatctacacccaccagagtgatgtgtggtctttcggggtgctcctgtgggagatcttcactctgggcggctccccataccccggtgtgcctgtggaggaacttttcaagctgctgaaggagggtcaccgcatggacaagcccagtaactgcaccaacgagctgtacatgatgatgcgggactgctggcatgcagtgccctcacagagacccaccttcaagcagctggtggaagacctggaccgcatcgtggccttgacctccaaccaggagtacctggacctgtccatgcccctggaccagtactcccccagctttcccgacacccggagctctacgtgctcctcaggggaggattccgtcttctctcatgagccgctgcccgaggagccctgcctgccccgacacccagcccagcttgccaatggcggactcaaacgccgctgactgccacccacacgccctccccagactccaccgtcagctgtaaccctcacccacagcccctgctgggcccaccacctgtccgtccctgtcccctttcctgctggcaggagccggctgcctaccaggggccttcctgtgtggcctgccttcaccccactcagctcacctctccctccacctcctctccacctgctggtgagaggtgcaaagaggcagatctttgctgccagccacttcatcccctcccagatgttggaccaacacccctccctgccaccaggcactgcctggagggcagggagtgggagccaatgaacaggcatgcaagtgagagcttcctgagctttctcctgtcggtttggtctgttttgccttcacccataagcccctcgcactctggtggcaggtgccttgtcctcagggctacagcagtagggaggtcagtgcttcgtgcctcgattgaaggtgacctctgccccagataggtggtgccagtggcttattaattccgatactagtttgctttgctgaccaaatgcctggtaccagaggatggtgaggcgaaggccaggttgggggcagtgttgtggccctggggcccagccccaaactgggggctctgtatatagctatgaagaaaacacaaagtgtataaatctgagtatatatttacatgtctttttaaaagggtcgttaccagagatttacccatcgggtaagatgctcctggtggctgggaggcatcagttgctatatattaaaaacaaaaaagaaaaaaaaggaaaatgtttttaaaaaggtcatatattttttgctacttttgctgttttatttttttaaattatgttctaaacctattttcagtttaggtccctcaataaaaattgctgctgcttcatttatctatgggctgtatgaaaagggtgggaatgtccactggaaagaagggacacccacgggccctggggctaggtctgtcccgagggcaccgcatgctcccggcgcaggttccttgtaacctcttcttcctaggtcctgcacccagacctcacgacgcacctcctgcctctccgctgcttttggaaagtcagaaaaagaagatgtctgcttcgagggcaggaaccccatccatgcagtagaggcgctgggcagagagtcaaggcccagcagccatcgaccatggatggtttcctccaaggaaaccggtggggttgggctggggagggggcacctacctaggaatagccacggggtagagctacagtgattaagaggaaagcaagggcgcggttgctcacgcctgtaatcccagcactttgggacaccgaggtgggcagatcacttcaggtcaggagtttgagaccagcctggccaacttagtgaaaccccatctctactaaaaatgcaaaaattatccaggcatggtggcacacgcctgtaatcccagctccacaggaggctgaggcagaatcccttgaagctgggaggcggaggttgcagtgagccgagattgcgccattgcactccagcctgggcaacagagaaaacaaaaaggaaaacaaatgatgaaggtctgcagaaactgaaacccagacatgtgtctgccccctctatgtgggcatggttttgccagtgcttctaagtgcaggagaacatgtcacctgaggctagttttgcattcaggtccctggcttcgtttcttgttggtatgcctccccagatcgtccttcctgtatccatgtgaccagactgtatttgttgggactgtcgcagatcttggcttcttacagttcttcctgtccaaactccatcctgtccctcaggaacggggggaaaattctccgaatgtttttggttttttggctgcttggaatttacttctgccacctgctggtcatcactgtcctcactaagtggattctggctcccccgtacctcatggctcaaactaccactcctcagtcgctatattaaagcttatattttgctggattactgctaaatacaaaagaaagttcaatatgttttcatttctgtagggaaaatgggattgctgctttaaatttctgagctagggattttttggcagctgcagtgttggcgactattgtaaaattctctttgtttctctctgtaaatagcacctgctaacattacaatttgtatttatgtttaaagaaggcatcatttggtgaacagaactaggaaatgaatttttagctcttaaaagcatttgctttgagaccgcacaggagtgtctttccttgtaaaacagtgatgataatttctgccttggccctaccttgaagcaatgttgtgtgaagggatgaagaatctaaaagtcttcataagtccttgggagaggtgctagaaaaatataaggcactatcataattacagtgatgtccttgctgttactactcaaatcacccacaaatttccccaaagactgcgctagctgtcaaataaaagacagtgaaattgacctgaaaaaaaaaaaaaaaaa

SEQ ID NO: 23 - DNA, 4654 bp, Artificial Sequence
  NM_000141.4 (FGFR2)
ggcggcggctggaggagagcgcggtggagagccgagcgggcgggcggcgggtgcggagcgggcgagggagcgcgcgcggccgccacaaagctcgggcgccgcggggctgcatgcggcgtacctggcccggcgcggcgactgctctccgggctggcgggggccggccgcgagccccgggggccccgaggccgcagcttgcctgcgcgctctgagccttcgcaactcgcgagcaaagtttggtggaggcaacgccaagcctgagtcctttcttcctctcgttccccaaatccgagggcagcccgcgggcgtcatgcccgcgctcctccgcagcctggggtacgcgtgaagcccgggaggcttggcgccggcgaagacccaaggaccactcttctgcgtttggagttgctccccgcaaccccgggctcgtcgctttctccatcccgacccacgcggggcgcggggacaacacaggtcgcggaggagcgttgccattcaagtgactgcagcagcagcggcagcgcctcggttcctgagcccaccgcaggctgaaggcattgcgcgtagtccatgcccgtagaggaagtgtgcagatgggattaacgtccacatggagatatggaagaggaccggggattggtaccgtaaccatggtcagctggggtcgtttcatctgcctggtcgtggtcaccatggcaaccttgtccctggcccggccctccttcagtttagttgaggataccacattagagccagaagagccaccaaccaaataccaaatctctcaaccagaagtgtacgtggctgcgccaggggagtcgctagaggtgcgctgcctgttgaaagatgccgccgtgatcagttggactaaggatggggtgcacttggggcccaacaataggacagtgcttattggggagtacttgcagataaagggcgccacgcctagagactccggcctctatgcttgtactgccagtaggactgtagacagtgaaacttggtacttcatggtgaatgtcacagatgccatctcatccggagatgatgaggatgacaccgatggtgcggaagattttgtcagtgagaacagtaacaacaagagagcaccatactggaccaacacagaaaagatggaaaagcggctccatgctgtgcctgcggccaacactgtcaagtttcgctgcccagccggggggaacccaatgccaaccatgcggtggctgaaaaacgggaaggagtttaagcaggagcatcgcattggaggctacaaggtacgaaaccagcactggagcctcattatggaaagtgtggtcccatctgacaagggaaattatacctgtgtagtggagaatgaatacgggtccatcaatcacacgtaccacctggatgttgtggagcgatcgcctcaccggcccatcctccaagccggactgccggcaaatgcctccacagtggtcggaggagacgtagagtttgtctgcaaggtttacagtgatgcccagccccacatccagtggatcaagcacgtggaaaagaacggcagtaaatacgggcccgacgggctgccctacctcaaggttctcaaggccgccggtgttaacaccacggacaaagagattgaggttctctatattcggaatgtaacttttgaggacgctggggaatatacgtgcttggcgggtaattctattgggatatcctttcactctgcatggttgacagttctgccagcgcctggaagagaaaaggagattacagcttccccagactacctggagatagccatttactgcataggggtcttcttaatcgcctgtatggtggtaacagtcatcctgtgccgaatgaagaacacgaccaagaagccagacttcagcagccagccggctgtgcacaagctgaccaaacgtatccccctgcggagacaggtaacagtttcggctgagtccagctcctccatgaactccaacaccccgctggtgaggataacaacacgcctctcttcaacggcagacacccccatgctggcaggggtctccgagtatgaacttccagaggacccaaaatgggagtttccaagagataagctgacactgggcaagcccctgggagaaggttgctttgggcaagtggtcatggcggaagcagtgggaattgacaaagacaagcccaaggaggcggtcaccgtggccgtgaagatgttgaaagatgatgccacagagaaagacctttctgatctggtgtcagagatggagatgatgaagatgattgggaaacacaagaatatcataaatcttcttggagcctgcacacaggatgggcctctctatgtcatagttgagtatgcctctaaaggcaacctccgagaatacctccgagcccggaggccacccgggatggagtactcctatgacattaaccgtgttcctgaggagcagatgaccttcaaggacttggtgtcatgcacctaccagctggccagaggcatggagtacttggcttcccaaaaatgtattcatcgagatttagcagccagaaatgttttggtaacagaaaacaatgtgatgaaaatagcagactttggactcgccagagatatcaacaatatagactattacaaaaagaccaccaatgggcggcttccagtcaagtggatggctccagaagccctgtttgatagagtatacactcatcagagtgatgtctggtccttcggggtgttaatgtgggagatcttcactttagggggctcgccctacccagggattcccgtggaggaactttttaagctgctgaaggaaggacacagaatggataagccagccaactgcaccaacgaactgtacatgatgatgagggactgttggcatgcagtgccctcccagagaccaacgttcaagcagttggtagaagacttggatcgaattctcactctcacaaccaatgaggaatacttggacctcagccaacctctcgaacagtattcacctagttaccctgacacaagaagttcttgttcttcaggagatgattctgttttttctccagaccccatgccttacgaaccatgccttcctcagtatccacacataaacggcagtgttaaaacatgaatgactgtgtctgcctgtccccaaacaggacagcactgggaacctagctacactgagcagggagaccatgcctcccagagcttgttgtctccacttgtatatatggatcagaggagtaaataattggaaaagtaatcagcatatgtgtaaagatttatacagttgaaaacttgtaatcttccccaggaggagaagaaggtttctggagcagtggactgccacaagccaccatgtaacccctctcacctgccgtgcgtactggctgtggaccagtaggactcaaggtggacgtgcgttctgccttccttgttaattttgtaataattggagaagatttatgtcagcacacacttacagagcacaaatgcagtatataggtgctggatgtatgtaaatatattcaaattatgtataaatatatattatatatttacaaggagttattttttgtattgattttaaatggatgtcccaatgcacctagaaaattggtctctctttttttaatagctatttgctaaatgctgttcttacacataatttcttaattttcaccgagcagaggtggaaaaatacttttgctttcagggaaaatggtataacgttaatttattaataaattggtaatatacaaaacaattaatcatttatagttttttttgtaatttaagtggcatttctatgcaggcagcacagcagactagttaatctattgcttggacttaactagttatcagatcctttgaaaagagaatatttacaatatatgactaatttggggaaaatgaagttttgatttatttgtgtttaaatgctgctgtcagacgattgttcttagacctcctaaatgccccatattaaaagaactcattcataggaaggtgtttcattttggtgtgcaaccctgtcattacgtcaacgcaacgtctaactggacttcccaagataaatggtaccagcgtcctcttaaaagatgccttaatccattccttgaggacagaccttagttgaaatgatagcagaatgtgcttctctctggcagctggccttctgcttctgagttgcacattaatcagattagcctgtattctcttcagtgaattttgataatggcttccagactctttggcgttggagacgcctgttaggatcttcaagtcccatcatagaaaattgaaacacagagttgttctgctgatagttttggggatacgtccatctttttaagggattgctttcatctaattctggcaggacctcaccaaaagatccagcctcatacctacatcagacaaaatatcgccgttgttccttctgtactaaagtattgtgttttgctttggaaacacccactcactttgcaatagccgtgcaagatgaatgcagattacactgatcttatgtgttacaaaattggagaaagtatttaataaaacctgttaatttttatactgacaataaaaatgtttctacagatattaatgttaacaagacaaaataaatgtcacgcaacttatttttttaataaaaaaaaaaaaaaa

SEQ ID NO: 24 - DNA, 4304 bp, Artificial Sequence
  NM_000142.4 (FGFR3)
gtcgcgggcagctggcgccgcgcggtcctgctctgccggtcgcacggacgcaccggcgggccgccggccggagggacggggcgggagctgggcccgcggacagcgagccggagcgggagccgcgcgtagcgagccgggctccggcgctcgccagtctcccgagcggcgcccgcctcccgccggtgcccgcgccgggccgtggggggcagcatgcccgcgcgcgctgcctgaggacgccgcggcccccgcccccgccatgggcgcccctgcctgcgccctcgcgctctgcgtggccgtggccatcgtggccggcgcctcctcggagtccttggggacggagcagcgcgtcgtggggcgagcggcagaagtcccgggcccagagcccggccagcaggagcagttggtcttcggcagcggggatgctgtggagctgagctgtcccccgcccgggggtggtcccatggggcccactgtctgggtcaaggatggcacagggctggtgccctcggagcgtgtcctggtggggccccagcggctgcaggtgctgaatgcctcccacgaggactccggggcctacagctgccggcagcggctcacgcagcgcgtactgtgccacttcagtgtgcgggtgacagacgctccatcctcgggagatgacgaagacggggaggacgaggctgaggacacaggtgtggacacaggggccccttactggacacggcccgagcggatggacaagaagctgctggccgtgccggccgccaacaccgtccgcttccgctgcccagccgctggcaaccccactccctccatctcctggctgaagaacggcagggagttccgcggcgagcaccgcattggaggcatcaagctgcggcatcagcagtggagcctggtcatggaaagcgtggtgccctcggaccgcggcaactacacctgcgtcgtggagaacaagtttggcagcatccggcagacgtacacgctggacgtgctggagcgctccccgcaccggcccatcctgcaggcggggctgccggccaaccagacggcggtgctgggcagcgacgtggagttccactgcaaggtgtacagtgacgcacagccccacatccagtggctcaagcacgtggaggtgaatggcagcaaggtgggcccggacggcacaccctacgttaccgtgctcaagacggcgggcgctaacaccaccgacaaggagctagaggttctctccttgcacaacgtcacctttgaggacgccggggagtacacctgcctggcgggcaattctattgggttttctcatcactctgcgtggctggtggtgctgccagccgaggaggagctggtggaggctgacgaggcgggcagtgtgtatgcaggcatcctcagctacggggtgggcttcttcctgttcatcctggtggtggcggctgtgacgctctgccgcctgcgcagcccccccaagaaaggcctgggctcccccaccgtgcacaagatctcccgcttcccgctcaagcgacaggtgtccctggagtccaacgcgtccatgagctccaacacaccactggtgcgcatcgcaaggctgtcctcaggggagggccccacgctggccaatgtctccgagctcgagctgcctgccgaccccaaatgggagctgtctcgggcccggctgaccctgggcaagccccttggggagggctgcttcggccaggtggtcatggcggaggccatcggcattgacaaggaccgggccgccaagcctgtcaccgtagccgtgaagatgctgaaagacgatgccactgacaaggacctgtcggacctggtgtctgagatggagatgatgaagatgatcgggaaacacaaaaacatcatcaacctgctgggcgcctgcacgcagggcgggcccctgtacgtgctggtggagtacgcggccaagggtaacctgcgggagtttctgcgggcgcggcggcccccgggcctggactactccttcgacacctgcaagccgcccgaggagcagctcaccttcaaggacctggtgtcctgtgcctaccaggtggcccggggcatggagtacttggcctcccagaagtgcatccacagggacctggctgcccgcaatgtgctggtgaccgaggacaacgtgatgaagatcgcagacttcgggctggcccgggacgtgcacaacctcgactactacaagaagacaaccaacggccggctgcccgtgaagtggatggcgcctgaggccttgtttgaccgagtctacactcaccagagtgacgtctggtcctttggggtcctgctctgggagatcttcacgctggggggctccccgtaccccggcatccctgtggaggagctcttcaagctgctgaaggagggccaccgcatggacaagcccgccaactgcacacacgacctgtacatgatcatgcgggagtgctggcatgccgcgccctcccagaggcccaccttcaagcagctggtggaggacctggaccgtgtccttaccgtgacgtccaccgacgagtacctggacctgtcggcgcctttcgagcagtactccccgggtggccaggacacccccagctccagctcctcaggggacgactccgtgtttgcccacgacctgctgcccccggccccacccagcagtgggggctcgcggacgtgaagggccactggtccccaacaatgtgaggggtccctagcagcccaccctgctgctggtgcacagccactccccggcatgagactcagtgcagatggagagacagctacacagagctttggtctgtgtgtgtgtgtgtgcgtgtgtgtgtgtgtgtgtgcacatccgcgtgtgcctgtgtgcgtgcgcatcttgcctccaggtgcagaggtaccctgggtgtccccgctgctgtgcaacggtctcctgactggtgctgcagcaccgaggggcctttgttctggggggacccagtgcagaatgtaagtgggcccacccggtgggacccccgtggggcagggagctgggcccgacatggctccggcctctgcctttgcaccacgggacatcacagggtgggcctcggcccctcccacacccaaagctgagcctgcagggaagccccacatgtccagcaccttgtgcctggggtgttagtggcaccgcctccccacctccaggctttcccacttcccaccctgcccctcagagactgaaattacgggtacctgaagatgggagcctttaccttttatgcaaaaggtttattccggaaactagtgtacatttctataaatagatgctgtgtatatggtatatatacatatatatatataacatatatggaagaggaaaaggctggtacaacggaggcctgcgaccctgggggcacaggaggcaggcatggccctgggcggggcgtgggggggcgtggagggaggccccagggggtctcacccatgcaagcagaggaccagggccttttctggcaccgcagttttgttttaaaactggacctgtatatttgtaaagctatttatgggcccctggcactcttgttcccacaccccaacacttccagcatttagctggccacatggcggagagttttaatttttaacttattgacaaccgagaaggtttatcccgccgatagagggacggccaagaatgtacgtccagcctgccccggagctggaggatcccctccaagcctaaaaggttgttaatagttggaggtgattccagtgaagatattttatttcctttgtcctttttcaggagaattagatttctataggatttttctttaggagatttattttttggacttcaaagcaagctggtattttcatacaaattcttctaattgctgtgtgtcccaggcagggagacggtttccagggaggggccggccctgtgtgcaggttccgatgttattagatgttacaagtttatatatatctatatatataatttattgagtttttacaagatgtatttgttgtagacttaacacttcttacgcaatgcttctagagttttatagcctggactgctacctttcaaagcttggagggaagccgtgaattcagttggttcgttctgtactgttactgggccctgagtctgggcagctgtcccttgcttgcctgcagggccatggctcagggtggtctcttcttggggcccagtgcatggtggccagaggtgtcacccaaaccggcaggtgcgattttgttaacccagcgacgaactttccgaaaaataaagacacctggttgctaacctggaaaaaaaaaaaaaaaaa

(19) United States
(12) Patent Application Publication
(10) Pub. No.: US 2018/0333418 A1
(43) Pub. Date: Nov. 22, 2018

(54) USE OF PAN FGFR INHIBITORS AND METHOD OF IDENTIFYING PATIENTS WITH CANCER ELIGIBLE FOR TREATMENT WITH A PAN FGFR INHIBITOR

(71) Applicant: BAYER PHARMA AKTIENGESELLSCHAFT, Berlin (DE)

(72) Inventors: Peter ELLINGHAUS, Melle (DE); Melanie HEROULT, Düsseldorf (DE)

(21) Appl. No.: 15/534,033

(22) PCT Filed: Dec. 8, 2015
(86) PCT No.: PCT/EP2015/078919

§ 371 Date: Jun. 8, 2017

(30) Foreign Application Priority Data
Dec. 11, 2014 (EP) 14197400.6

(51) Int. Cl. A61K 31/53; C12Q 1/6886; A61K 31/506; A61K 31/496; A61K 31/498; A61K 31/4184; A61P 35/00

(52) U.S. Cl. CPC A61K 31/53; C12Q 1/6886; A61K 31/506; C12Q 2600/106; A61K 31/498; A61K 31/4184; A61P 35/00; A61K 31/496

(57) ABSTRACT

The current invention is based on a pan FGFR inhibitor for use in the treatment of cancer in a subject, wherein the subject is one for whom the sum of FGFR1, FGFR2 and/or FGFR3 mRNA in a tumor tissue sample from the subject has been found to be overexpressed.

The current invention is based on a pan FGFR inhibitor for use in the treatment of cancer in a subject, wherein the subject is one for whom the sum of FGFR1, FGFR2 and/or FGFR3 mRNA in a tumor tissue sample from the subject has been found to be overexpressed

In a further embodiment the invention is directed to a method of identifying patients with cancer eligible for treatment with a pan FGFR inhibitor comprising testing a tumor tissue sample from the patient for the presence of FGFR1, FGFR2 and/or FGFR3 mRNA overexpression, wherein the patient is eligible for treatment with a pan FGFR inhibitor if the sum of the measured mRNA expression of FGFR1, FGFR2 and FGFR3 is overexpressed.

Cancer is a leading cause of death worldwide and accounted for 7.6 million deaths (around 13% of all deaths) in 2008. Deaths from cancer are projected to continue to rise worldwide to over 11 million in 2030 (WHO source, Fact Sheet No. 297, February 2011).

There are many ways how cancers can arise which is one of the reasons why their therapy is difficult. One way that transformation of cells can occur is following a genetic alteration. The completion of the human genome project showed genomic instability and heterogeneity of human cancer genes. Recent strategies to identify these genetic alterations sped up the process of cancer-gene discovery. Gene abnormality can, for instance, lead to the overexpression of proteins, and hence to a non-physiological activation of these proteins. One family of proteins from which a number of oncoproteins derive are tyrosine kinases and in particular receptor tyrosine kinases (RTKs). In the past two decades, numerous avenues of research have demonstrated the importance of RTK-mediated signalling in adverse cell growth leading to cancer. In recent years, promising results have been achieved in the clinic with selective small-molecule inhibitors of tyrosine kinases as a new class of anti-tumorigenic agents [Swinney and Anthony, Nature Rev. Drug Disc. 10 (7), 507-519 (2011)].

Fibroblast growth factors (FGFs) and their receptors (FGFRs) form part of a unique and diverse signalling system which plays a key role in a variety of biological processes which encompass various aspects of embryonic development and adult pathophysiology [Itoh and Ornitz, J. Biochem. 149 (2), 121-130 (2011)]. In a spatio-temporal manner, FGFs stimulate through FGFR binding a wide range of cellular functions including migration, proliferation, differentiation, and survival.

The FGF family comprises 18 secreted polypeptidic growth factors that bind to four highly conserved receptor tyrosine kinases (FGFR-1 to -4) expressed at the cell surface. In addition, FGFR-5 can bind to FGFs but does not have a kinase domain, and therefore is devoid of intracellular signalling. The specificity of the ligand/receptor interaction is enhanced by a number of transcriptional and translational processes which give rise to multiple isoforms by alternative transcriptional initiation, alternative splicing, and C-terminal truncations. Various heparan sulfate proteoglycans (e.g. syndecans) can be part of the FGF/FGFR complex and strongly influence the ability of FGFs to induce signalling responses [Polanska et al., Developmental Dynamics 238 (2), 277-293 (2009)]. FGFRs are cell surface receptors consisting of three extracellular immunoglobulin-like domains, a single-pass transmembrane domain, and an intracellular dimerized tyrosine kinase domain. Binding of FGF bring the intracellular kinases into close proximity, enabling them to transphosphorylate each other. Seven phosphorylation sites have been identified (e.g., in FGFR-1 Tyr463, Tyr583, Tyr585, Tyr653, Tyr654, Tyr730, and Tyr766).

Some of these phosphotyrosine groups act as docking sites for downstream signalling molecules which themselves may also be directly phosphorylated by FGFR, leading to the activation of multiple signal transduction pathways. Thus, the MAPK signalling cascade is implicated in cell growth and differentiation, the PI3K/Akt signalling cascade is involved in cell survival and cell fate determination, while the PI3K and PKC signalling cascades have a function in the control of cell polarity. Several feedback inhibitors of FGF signalling have now been identified and include members of the Spry (Sprouty) and Sef (similar expression to FGF) families. Additionally, in certain conditions, FGFR is released from pre-Golgi membranes into the cytosol. The receptor and its ligand, FGF-2, are co-transported into the nucleus by a mechanism that involves importin, and are engaged in the CREB-binding protein (CBP) complex, a common and essential transcriptional co-activator that acts as a gene activation gating factor. Multiple correlations between the immunohistochemical expression of FGF-2, FGFR-1 and FGFR-2 and their cytoplasmic and nuclear tumor cell localizations have been observed. For instance, in lung adenocarcinomas this association is also found at the nuclear level, emphasizing an active role of the complex at the nucleus [Korc and Friesel, Curr. Cancer Drugs Targets 5, 639-651 (2009)].

FGFs are widely expressed in both developing and adult tissues and play important roles in a variety of normal and pathological processes, including tissue development, tissue regeneration, angiogenesis, neoplastic transformation, cell migration, cellular differentiation, and cell survival. Additionally, FGFs as pro-angiogenic factors have also been implicated in the emerging phenomenon of resistance to vascular endothelial growth factor receptor-2 (VEGFR-2) inhibition [Bergers and Hanahan, Nat. Rev. Cancer 8, 592-603 (2008)].

Recent oncogenomic profiles of signalling networks demonstrated an important role for aberrant FGF signalling in the emergence of some common human cancers [Wesche et al., Biochem. J. 437 (2), 199-213 (2011)]. Ligand-independent FGFR constitutive signalling has been described in many human cancers, such as brain cancer, head and neck cancer, gastric cancer and ovarian cancer. FGFR-mutated forms as well as FGFR-intragenic translocations have been identified in malignancies such as myeloproliferative diseases. Interestingly, the same mutations discovered to be the cause of many developmental disorders are also found in tumor cells (e.g., the mutations found in achondroplasia and thanatophoric dysplasia, which cause dimerization and thus constitutive activation of FGFR-3, are also frequently found in bladder cancer). A mutation that promotes dimerization is just one mechanism that can increase ligand-independent signalling from FGFRs. Other mutations located inside or outside of the kinase domain of FGFRs can change the conformation of the domain giving rise to permanently active kinases.

Amplification of the chromosomal region 8p11-12, the genomic location of FGFR-1, is a common focal amplification in breast cancer and occurs in approximately 10% of breast cancers, predominantly in oestrogen receptor-positive cancers. FGFR-1 amplifications have also been reported in non-small cell lung squamous carcinoma and are found at a low incidence in ovarian cancer, bladder cancer and rhabdomyosarcoma. Similarly, approximately 10% of gastric cancers show FGFR-2 amplification, which is associated with poor squameous non-small cell lung cancer (sqNSCLC) prognosis, diffuse-type cancers. Moreover, multiple single nucleotide polymorphisms (SNPs) located in FGFR-1 to -4 were found to correlate with an increased risk of developing selective cancers, or were reported to be associated with poor prognosis (e.g., FGFR-4 G388R allele in breast cancer, colon cancer and lung adenocarcinoma). The direct role of these SNPs to promote cancer is still controversial.

Amplification of the FGFR1 8p12 gene locus has been observed in up to 20% of subjects [Dutt A. et al. PLoS One. 2011; 6(6):e20351]. FGFR1 gene amplification is so far one of the most frequently observed molecular alteration in sqNSCLC whereas mutations in FGFR-encoding genes are rather rare in sqNSCLC subjects (<2%) [Lim et al. Future Oncol. 2013 March; 9(3):377-86]. Recent publications investigating the correlation between FGFR1 gene amplification and target expression level (mRNA or protein expression) revealed a very high proportion of sqNSCLC subjects (50%) that do show a high FGFR1 mRNA overexpression in tumor tissue in the absence of a FGFR1 gene amplification. Even more important, in 54% of subjects with a confirmed FGFR1 copy number gain, this gain does not lead to higher FGFR1 target expression levels making a treatment success with a pan-FGFR inhibitor very unlikely. Furthermore, high FGFR1 mRNA expression could also be observed in 22% of lung adenocarcinomas (AC), whereas no single case of FGFR1 amplification was found in this histology type. In vitro, FGFR1 mRNA expression in lung cancer cell lines correlated better with the antiproliferative response to the FGFR inhibitor ponatinib, than the FGFR1 copy number of the respective cell line [Wynes et al.].

FGFR1 gene amplification is observed in 12.6% of cases in squamous cell carcinoma of the nead & neck (HNSCC) [Boehm D. et al. Virchows Arch. 2014 May; 464(5):547-51] whereas the prevalence of FGFR1 tumor protein overexpression in the literature ranges from 12 to 100%. Prevalence of FGFR1 mRNA overexpression in HNSCC patient tumors has not been examined in the literature so far, but a recent publication characterized SCC (squamous cell carcinoma) cell lines of the head and neck region according to their FGFR1 copy number, mRNA and protein expression status and subsequently tested their sensitivity towards the small molecule FGFR inhibitor BGJ398. The authors found FGFR1 gene amplification neither correlating with mRNA nor with protein expression. Interestingly, sensitivity to BGJ398 was only observed in those cell lines harboring high protein and mRNA levels [Maessenhausen et al. Annals of Translational Medicine Vol 1, No 3 (October 2013)]. Nothing is known about the prevalence of FGFR2 mRNA overexpression in primary HNSCC tumors and its correlation to response-to-treatment to an FGFR inhibitor, whereas activating mutations in FGFR2-encoding gene rendered a patient-derived HNSCC cell line sensitive towards treatment with an FGFR inhibitor [Liao, R G Cancer Res. 2013 Aug. 15; 73(16):5195-205]. Regarding FGFR3 mRNA expression in HNSCC, a recent publication observed rather a lower expression of FGFR3 mRNA in HNSCC cancer patient tumors when compared to non-tumor controls [Marshall M E et al. Clin Cancer Res. 2011 Aug. 1; 17(15):5016-25]. No correlation between FGFR3 mRNA expression in HNSCC and response-to-treatment with an FGFR inhibitor has been described so far.

FGFR1 gene is amplified in about 21% of esophageal cancer patients [Bandla et al, Ann Thorac Surg. 2012 April; 93(4):1101-6] whereas FGFR2 gene is amplified in about 4% of esophageal cancer patients [Kato H et al. Int J Oncol. 2013 April; 42(4):1151-8]. FGFR1 [De-Chen, L VOLUME 46|NUMBER 5|MAY 2014 Nature Genetics] and FGFR2 [Paterson et al. J Pathol. 2013 May; 230(1):118-28] proteins have been found to be overexpressed in 10-20% of esophageal cancer patients. FGFR1 and FGFR2 mRNA expression levels have not been investigated in esophageal cancer patients so far. Nothing is known about drug sensitivity of esophageal cancer to pan-FGFR inhibitors.

FGFR1 amplification has been observed in about 8% of ovarian cancers [Theillet et al. Genes Chromosom. Cancer, 7: 219-226] and FGFR2 overexpression was recently observed [Taniguchi et al. Int J Gynecol Cancer. 2013 June; 23(5):791-6]. Regarding drug sensitivity towards FGFR inhibitors, the ovarian cancer cell line A2780 was found to be sensitive in vitro towards treatment with BGJ398 [Guagnano et al. Cancer Discov. 2012 December; 2(12):1118-33], In contrast, a FGFR2 fusion in an ovarian cancer patient rendered her circulating tumor cells sensitive to BGJ398 treatment [Martignetti et al. Neoplasia. 2014 January; 16(1):97-103]. So the oncogenic driver function of DNA alterations in FGFR-encoding genes remains controversial.

FGFR1 amplification is observed in about 18% of osteosarcoma patients [Fernanda-Amary et al., Cancer Med. 2014 August; 3(4):980-7] and in line with that, anti-proliferative effects of the FGFR small-molecule inhibitor BGJ398 were observed in the FGFR1-amplified osteosarcoma cell line G-292 [see Guagnano et al].

In summary, a great number of in vitro and in vivo studies have been performed that validate FGFR-1 to -4 as important cancer targets, and comprehensive reviews have summarized these findings [see, for example, Heinzle et al., Expert Opin. Ther. Targets 15 (7), 829-846 (2011); Wesche et al., Biochem. J. 437 (2), 199-213 (2011); Greulich and Pollock, Trends in Molecular Medicine 17 (5), 283-292 (2011); Haugsten et al., Mol. Cancer Res. 8 (11), 1439-1452 (2010)]. Several strategies have been followed to attenuate aberrant FGFR-1 to -4 signalling in human tumors including blocking antibodies and small-molecule inhibitors, amongst others. A number of selective small-molecule FGFR inhibitors are currently in clinical development, such as AZD-4547 (AstraZeneca Compound of formula (III)), BJG-398 (Novartis, compound of formula (II)) JNJ-42756493 (Johnson&Johnson, compound of formula (IV)) and CH 5183284 (Chanugi, compound of formula (V)).

While only cancer patients are being tested and enrolled in FGFR tyrosine kinase inhibitor (TKI) clinical trials based on a) elevated FGFR1 or FGFR2 gene copy number, b) activating mutations in FGFR-encoding genes or c) the occurrence of FGFR fusion proteins [Wynes et al. Clin Cancer Res. 2014 Jun. 15; 20(12):3299-309]

Here, we identified that the sum of FGFR1, FGFR2 and/or FGFR3 mRNA expression is especially suited to predict treatment response to pan FGFR inhibitors according to formula (I):

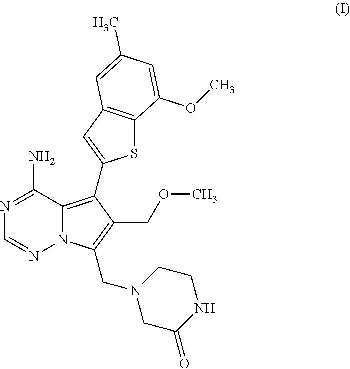 [SMILES: COCc1c(-c2cc3cc(C)cc(OC)c3s2)c2c(N)ncnn2c1CN1CCNC(=O)C1]

which can be present in form of its salt, solvate and/or solvates of the salt.

Further preferred panFGFR inhibitors according to this invention are for example AZD-4547 (AstraZeneca, compound of formula (III)), BJG-398 (Novartis, compound of formula (II)), JNJ-42756493 (Johnson&Johnson, compound of formula (IV)) and CH 5183284 (Chanugi, compound of formula (V)) all of them can be present in form of their salt, solvate and/or solvates of the salt

Salts for the purposes of the present invention are preferably pharmaceutically acceptable salts of the compounds according to the invention (for example, see S. M. Berge et al., “Pharmaceutical Salts”, J. Pharm. Sci. 1977, 66, 1-19). Salts which are not themselves suitable for pharmaceutical uses but can be used, for example, for isolation or purification of the compounds according to the invention are also included.

Pharmaceutically acceptable salts include acid addition salts of mineral acids, carboxylic acids and sulfonic acids, for example salts of hydrochloric acid, hydrobromic acid, sulfuric acid, phosphoric acid, methanesulfonic acid, ethanesulfonic acid, benzenesulfonic acid, toluenesulfonic acid, naphthalenedisulfonic acid, formic acid, acetic acid, trifluoroacetic acid, propionic acid, lactic acid, tartaric acid, malic acid, citric acid, fumaric acid, maleic acid, and benzoic acid.

Pharmaceutically acceptable salts also include salts of customary bases, such as for example and preferably alkali metal salts (for example sodium and potassium salts), alkaline earth metal salts (for example calcium and magnesium salts), and ammonium salts derived from ammonia or organic amines, such as illustratively and preferably ethylamine, diethylamine, triethylamine, N,N-diiso-propylethylamine, monoethanolamine, diethanolamine, triethanolamine, dimethylaminoethanol, diethylaminoethanol, procaine, dicyclohexylamine, dibenzylamine, N-methylmorpholine, N-methylpiperidine, arginine, lysine, and 1,2-ethylenediamine.

Solvates in the context of the invention are designated as those forms of the compounds according to the invention which form a complex in the solid or liquid state by stoichiometric coordination with solvent molecules. Hydrates are a specific form of solvates, in which the coordination takes place with water. Hydrates are preferred solvates in the context of the present invention.

The current invention is based on a pan FGFR inhibitor for use in the treatment of cancer in a subject, wherein the subject is one for whom the sum of FGFR1, FGFR2 and/or FGFR3 mRNA in a tumor tissue sample from the subject has been found to be overexpressed.

In another embodiment the invention relates to a method of identifying patients with cancer eligible for treatment with a pan FGFR inhibitor comprising testing a tumor tissue sample from the patient for the presence of FGFR1, FGFR2 and/or FGFR3 mRNA overexpression, wherein the patient is eligible for treatment with a pan FGFR inhibitor if the sum of the measured mRNA expression of FGFR1, FGFR2 and FGFR3 is overexpressed.

Cancer according to the current invention are cancer and tumor diseases. These are understood as meaning, in particular, the following diseases, but without being limited to them: mammary carcinomas and mammary tumors (ductal and lobular forms, also in situ), tumors of the respiratory tract (small cell and non-small cell lung carcinoma (NSCLC), NSCLC includes lung adenocarcinoma of the lung, squameous cell lung carcinoma and large-cell lung carcinoma, parvicellular and non-parvicellular carcinoma, bronchial carcinoma, bronchial adenoma, pleuropulmonary blastoma), cerebral tumors (e.g. of the brain stem and of the hypothalamus, astrocytoma, glioblastoma, medulloblastoma, ependymoma, and neuro-ectodermal and pineal tumors), tumors of the digestive organs (oesophagus, stomach, gall bladder, small intestine, large intestine, rectum, anus), liver tumors (inter alia hepatocellular carcinoma, cholangiocellular carcinoma and mixed hepatocellular and cholangiocellular carcinoma), tumors of the head and neck region (larynx, hypopharynx, nasopharynx, oropharynx, lips and oral cavity), skin tumors (squamous epithelial carcinoma, Kaposi sarcoma, malignant melanoma, Merkel cell skin cancer and non-melanomatous skin cancer), tumors of soft tissue (inter alia soft tissue sarcomas, osteosarcomas, malignant fibrous histiocytomas, lymphosarcomas and rhabdomyosarcomas), tumors of the eyes (inter alia intraocular melanoma, uveal melanoma and retinoblastoma), tumors of the endocrine and exocrine glands (e.g. thyroid and parathyroid glands, pancreas and salivary gland), tumors of the urinary tract (tumors of the bladder, penis, kidney, renal pelvis and ureter), tumors of the reproductive organs (carcinomas of the endometrium, cervix, ovary, vagina, vulva and uterus in women, and carcinomas of the prostate and testicles in men), as well as distant metastases thereof. These disorders also include proliferative blood diseases in solid form and as circulating blood cells, such as lymphomas, leukaemias and myeloproliferative diseases, e.g. acute myeloid, acute lymphoblastic, chronic lymphocytic, chronic myelogenic and hairy cell leukaemia, and AIDS-related lymphomas, Hodgkin's lymphomas, non-Hodgkin's lymphomas, cutaneous T-cell lymphomas, Burkitt's lymphomas, and lymphomas in the central nervous system.

In a preferred embodiment cancer according to this invention is head and neck, preferably squameous cell carcinoma of head and neck, esophageal cancer, ovarian cancer, bladder cancer, colon cancer and/or lung cancer. In an even more preferred embodiment lung cancer according to this invention is NSCLC, more preferred squameous cell carcinoma of the lung.

In an other preferred embodiment sarcoma according to this invention can be liposarcoma, fibrosarcoma, leiomyosarcoma, chondrosarcoma, synovialsarcoma, angiosarcoma, ewingsarcoma and clear-cell-sarcoma.

In a preferred embodiment the pan FGFR inhibitor is selected from the group consisting of compounds of formula (I), (II), (III), (IV) and/or (V) which can be present in form of their salt, solvate and/or solvates of the salt:

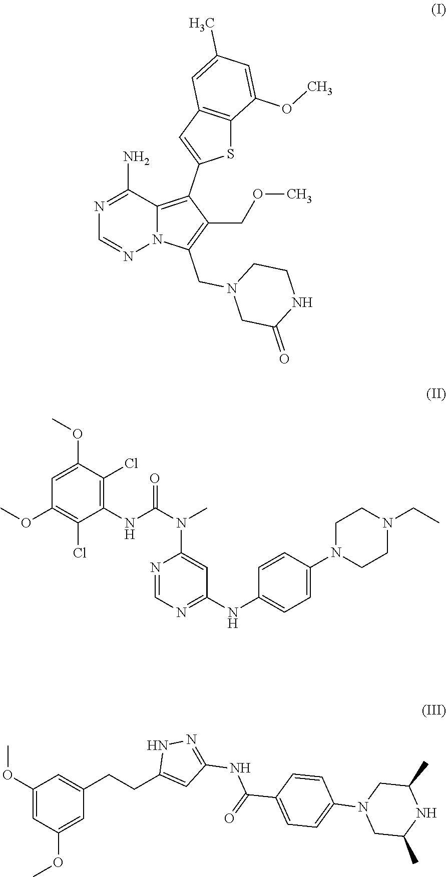 [SMILES: COCc1c(-c2cc3cc(C)cc(OC)c3s2)c2c(N)ncnn2c1CN1CCNC(=O)C1 ; CCN1CCN(c2ccc(Nc3cc(N(C)C(=O)Nc4c(Cl)c(OC)cc(OC)c4Cl)ncn3)cc2)CC1 ; COc1cc(CCc2cc(NC(=O)c3ccc(N4C[C@@H](C)N[C@@H](C)C4)cc3)n[nH]2)cc(OC)c1]

In an especially preferred embodiment the pan FGFR inhibitor according to the current invention is the inhibitor of formula (I).

Compounds of formula (I), (II), (III), (IV) and/or (V), may be administered as the sole pharmaceutical agent or in combination with one or more additional therapeutic agents as long as this combination does not lead to undesirable and/or unacceptable side effects. Such combination therapy includes administration of a single pharmaceutical dosage formulation which contains a compound of formula (I), (II), (III), (IV) and/or (V), as defined above, and one or more additional therapeutic agents, as well as administration of a compound of formula (I)), (II), (III), (IV) and/or (V), and each additional therapeutic agent in its own separate pharmaceutical dosage formulation. For example, a compound of formula (I), (II), (III), (IV) and/or (V), and a therapeutic agent may be administered to the patient together in a single (fixed) oral dosage composition such as a tablet or capsule, or each agent may be administered in separate dosage formulations.

Where separate dosage formulations are used, the compound of formula (I)), (II), (III), (IV) and/or (V), and one or more additional therapeutic agents may be administered at essentially the same time (i.e., concurrently) or at separately staggered times (i.e., sequentially).

In particular, the compounds of formula (I), (II), (III), (IV) and/or (V), may be used in fixed or separate combination with other anti-cancer agents such as alkylating agents, anti-metabolites, plant-derived anti-tumor agents, hormonal therapy agents, topoisomerase inhibitors, tubulin inhibitors, kinase inhibitors, targeted drugs, antibodies, antibody-drug conjugates (ADCs), immunologicals, biological response modifiers, anti-angiogenic compounds, and other anti-proliferative, cytostatic and/or cytotoxic substances. In this regard, the following is a non-limiting list of examples of secondary agents that may be used in combination with the compounds of the present invention:

Abarelix, abiraterone, aclarubicin, afatinib, aflibercept, aldesleukin, alemtuzumab, alitretinoin, alpharadin, altretamine, aminoglutethimide, amonafide, amrubicin, amsacrine, anastrozole, andromustine, arglabin, asparaginase, axitinib, 5-azacitidine, basiliximab, belotecan, bendamustine, bevacizumab, bexarotene, bicalutamide, bisantrene, bleomycin, bortezomib, bosutinib, brivanib alaninate, buserelin, busulfan, cabazitaxel, CAL-101, calcium folinate, calcium levofolinate, camptothecin, capecitabine, carboplatin, carmofur, carmustine, catumaxomab, cediranib, celmoleukin, cetuximab, chlorambucil, chlormadinone, chlormethine, cidofovir, cisplatin, cladribine, clodronic acid, clofarabine, combretastatin, crisantaspase, crizotinib, cyclophosphamide, cyproterone, cytarabine, dacarbazine, dactinomycin, darbepoetin alfa, darinaparsin, dasatinib, daunorubicin, decitabine, degarelix, denileukin diftitox, denosumab, deslorelin, dibrospidium chloride, docetaxel, dovitinib, doxifluridine, doxorubicin, dutasteride, eculizumab, edrecolomab, eflornithine, elliptinium acetate, eltrombopag, endostatin, enocitabine, epimbicin, epirubicin, epitiostanol, epoetin alfa, epoetin beta, epothilone, eptaplatin, eribulin, erlotinib, estradiol, estramustine, etoposide, everolimus, exatecan, exemestane, exisulind, fadrozole, fenretinide, filgrastim, finasteride, flavopiridol, fludarabine, 5-fluorouracil, fluoxymesterone, flutamide, foretinib, formestane, fotemustine, fulvestrant, ganirelix, gefitinib, gemcitabine, gemtuzumab, gimatecan, gimeracil, glufosfamide, glutoxim, goserelin, histrelin, hydroxyurea, ibandronic acid, ibritumomab tiuxetan, idarubicin, ifosfamide, imatinib, imiquimod, improsulfan, intedanib, interferon alpha, interferon alpha-2a, interferon alpha-2b, interferon beta, interferon gamma, interleukin-2, ipilimumab, irinotecan, ixabepilone, lanreotide, lapatinib, lasofoxifene, lenalidomide, lenograstim, lentinan, lenvatinib, lestaurtinib, letrozole, leuprorelin, levamisole, linifanib, linsitinib, lisuride, lobaplatin, lomustine, lonidamine, lurtotecan, mafosfamide, mapatumumab, masitinib, masoprocol, medroxyprogesterone, megestrol, melarsoprol, melphalan, mepitiostane, mercaptopurine, methotrexate, methyl aminolevulinate, methyltestosterone, mifamurtide, mifepristone, miltefosine, miriplatin, mitobronitol, mitoguazone, mitolactol, mitomycin, mitotane, mitoxantrone, molgramostim, motesanib, nandrolone, nedaplatin, nelarabine, neratinib, nilotinib, nilutamide, nimotuzumab, nimustine, nitracrine, nolatrexed, ofatumumab, oprelvekin, oxaliplatin, paclitaxel, palifermin, pamidronic acid, panitumumab, pazopanib, pegaspargase, peg-epoetin beta, pegfilgastrim, peg-interferon alpha-2b, pelitrexol, pemetrexed, pemtumomab, pentostatin, peplomycin, perfosfamide, perifosine, pertuzumab, picibanil, pirambicin, pirarubicin, plerixafor, plicamycin, poliglusam, polyestradiol phosphate, ponatinib, porfimer sodium, pralatrexate, prednimustine, procarbazine, procodazole, PX-866, quinagolide, raloxifene, raltitrexed, ranibizumab, ranimustine, razoxane, regorafenib, risedronic acid, rituximab, romidepsin, romiplostim, rubitecan, saracatinib, sargramostim, satraplatin, selumetinib, sipuleucel-T, sirolimus, sizofiran, sobuzoxane, sorafenib, streptozocin, sunitinib, talaporfin, tamibarotene, tamoxifen, tandutinib, tasonermin, teceleukin, tegafur, telatinib, temoporfin, temozolomide, temsirolimus, teniposide, testolactone, testosterone, tetrofosmin, thalidomide, thiotepa, thymalfasin, tioguanine, tipifarnib, tivozanib, toceranib, tocilizumab, topotecan, toremifene, tositumomab, trabectedin, trastuzumab, treosulfan, tretinoin, triapine, trilostane, trimetrexate, triptorelin, trofosfamide, ubenimex, valrubicin, vandetanib, vapreotide, varlitinib, vatalanib, vemurafenib, vidarabine, vinblastine, vincristine, vindesine, vinflunine, vinorelbine, volociximab, vorinostat, zinostatin, zoledronic acid, and zorubicin.

Generally, the following aims may be pursued with the combination of compounds of formula (I), (II), (III), (IV) and/or (V), with other anti-cancer agents:
improved activity in slowing down the growth of a tumor, in reducing its size or even in its complete elimination compared with treatment with a single active compound;
possibility of employing the chemotherapeutics used in a lower dosage than in monotherapy;
possibility of a more tolerable therapy with few side effects compared with individual administration;
possibility of treatment of a broader spectrum of cancer and tumor diseases;
achievement of a higher rate of response to therapy;
longer survival time of the patient compared with standard therapy.

In cancer treatment, the compounds of formula (I), (II), (III), (IV) and/or (V), may also be employed in conjunction with radiation therapy and/or surgical intervention.

As used herein, an “FGF receptor” is a receptor protein tyrosine kinase which belongs to the FGF receptor family and includes FGFR1, FGFR2, FGFR3 FGFR4 and other members of this family to be identified in the future. The FGF receptor will generally comprise an extracellular domain, which may bind an FGF ligand; a lipophilic transmembrane domain; a conserved intracellular tyrosine kinase domain; and a carboxyl-terminal signaling domain harboring several tyrosine residues which can be phosphorylated. The FGF receptors may be a native sequence FGF receptor or an amino acid sequence variant thereof. Preferably the FGF receptor is native sequence human FGF receptor.

By “tissue sample” according to the current invention is meant a collection of similar cells obtained from a tissue of a subject or patient, preferably containing nucleated cells with chromosomal material. The four main human tissues are (1) epithelium; (2) the connective tissues, including blood vessels, bone and cartilage; (3) muscle tissue; and (4) nerve tissue. The source of the tissue sample may be solid tissue as from a fresh, frozen and/or preserved organ or tissue sample or biopsy

For the purposes herein a “section” of a tissue sample is meant a single part or piece of a tissue sample, e.g., a thin slice of tissue or cells cut from a tissue sample. It is understood that multiple sections of tissue samples may be taken and subjected to analysis according to the present invention.

### Sample Preparation

Any tissue sample from a subject may be used. Examples of tissue samples that may be used include, but are not limited to ovary, lung, endometrium, head, neck, esophageal and bladder. The tissue sample can be obtained by a variety of procedures including, but not limited to surgical excision, or biopsy. The tissue may be fresh or frozen. In one embodiment, the tissue sample is fixed and embedded in paraffin or the like.

The tissue sample may be fixed (i.e., preserved) by conventional methodology (See e.g., Manual of Histological Staining Method of the Armed Forces Institute of Pathology, 3rd Edition Lee G. Luna, H T (ASCP) Editor, The Blakston Division McGraw-Hill Book Company: New York; (1960); The Armed Forces Institute of Pathology Advanced Laboratory Methods in Histology and Pathology (1994) Ulreka V. Mikel, Editor, Armed Forces Institute of Pathology, American Registry of Pathology, Washington, D.C.). One of skill in the art will appreciate that the choice of a fixative is determined by the purpose for which the tissue is to be histologically stained or otherwise analyzed. One of skill in the art will also appreciate that the length of fixation depends upon the size of the tissue sample, and the fixative used. By way of example, neutral buffered formalin, may be used to fix a tissue sample.

Generally, the tissue sample is first fixed and is then dehydrated through an ascending series of alcohols, infiltrated, and embedded with paraffin or other sectioning media so that the tissue sample may be sectioned. Alternatively, one may section the tissue and fix the sections obtained. By way of example, the tissue sample may be embedded and processed in paraffin by conventional methodology. Examples of paraffin that may be used include, but are not limited to, Paraplast, Broloid, and Tissuemay. Once the tissue sample is embedded, the sample may be sectioned by a microtome or the like. By way of example for this procedure, sections may range from about three microns to about five microns in thickness. Once sectioned, the sections may be attached to slides by several standard methods. Examples of slide adhesives include, but are not limited to, silane, gelatin, poly-L-lysine, and the like. Especially suitable for RNA in situ hydridization are, the paraffin embedded sections attached to positively charged slides, e.g. slides coated with poly-L-lysine.

If paraffin has been used as the embedding material, the tissue sections are generally deparaffinized and rehydrated to water. The tissue sections may be deparaffinized by several conventional standard methodologies. For example, xylenes and a gradually descending series of alcohols may be used. Alternatively, commercially available deparaffinizing non-organic agents such as Hemo-De7 (CMS, Houston, Tex.) may be used.

mRNA-Overexpression according to the current invention refers to a FGFR protein-encoding messenger RNA that is expressed at a higher level on tumor cells compared to normal cells. Generally, the normal cells for comparison are of the same tissue type, particularly phenotype, as the tumor, or from which the tumor arose.

FGFR1, 2 and 3 mRNA expression levels in tumor tissue samples are quantified by RNA in situ hybridization using FGFR1, 2 or 3 probes. Methods for in situ hybridization are known in the art e.g. as described by Wang et al in J Mol Diagn. 2012 January; 14(1):22-9. ISH probes to detect FGFR1, FGFR2 or FGFR3 mRNA expression are designed for example according to Jin and Lloyd (J Clin Lab Anal. 1997; 11(1):2-9.). Sequences used to design probes according to the present invention are sequences having GenBank sequence accession numbers NM_023110.2 (FGFR1), NM_000141.4 (FGFR2), or NM_000142.4 (FGFR3), whereas the person skilled in the art knows that the polyA tail as provided in the GenBank accession numbers above is not used for probe design. Methods are known for formalin-fixed, paraffin-embedded tissue specimens or frozen specimens. One can use either conventional chromogenic dyes for bright-field microscopy or fluorescent dyes for multiplex analysis. Levsky and Singer discuss developments in fluorescence in situ hybridization in J Cell Sci. 2003 Jul. 15; 116(Pt 14):2833-8.

Preferably, FGFR1, 2 and/or- 3 mRNA expression levels in tumor tissue samples are quantified by RNA in situ hybridization using RNAscope technology from ACD (Advanced Cell Diagnostics, Inc., 3960 Point Eden Way, Hayward, Calif. 94545, USA), preferably by using the FGFR1 probe catalogue #310071, FGFR2 probe catalogue #311171, and FGFR3 probe catalogue #310791.

### In Situ Hybridization (ISH)

In situ hybridization is commonly carried out on cells or tissue sections fixed to slides. Generally, in in situ procedures, direct and indirect methods are employed.

In the direct method, the detectable molecule (e.g. a fluorophore, i.e Fluorescence In Situ Hybridization or FISH) is bound directly to the nucleic acid probe so that probe-target hybrids can be visualized under a microscope immediately after the hybridization reaction. For such methods it is essential that the probe-reporter bond survives the rather harsh hybridization and washing conditions. Perhaps more important, however, is, that the reporter molecule does not interfere with the hybridization reaction. The terminal fluorochrome labeling procedure of RNA probes developed by Bauman et al. (1980, 1984), and the direct enzyme labeling procedure of nucleic acids described by Renz and Kurz (1984) meet these criteria. Boehringer Mannheim has introduced several fluorochrome-labeled nucleotides that can be used for labeling and direct detection of DNA or RNA probes. Alternatively, radioactive labeling can be used. Indirect procedures require the probe to contain a detectable molecule, introduced chemically or enzymatically, that can be detected by affinity cytochemistry, e.g. the biotin-streptavidin system.

E.g. fluorophores are used to label a nucleic acid sequence probe that is complementary to a target nucleotide sequence in the cell. Each cell containing the target nucleotide sequence will bind the labeled probe producing a fluorescence signal. The target nucleotide sequence is a FGFR1, FGFR2 or FGFR3 sequence. ISH analysis can be used in conjunction with other assays, including without limitation morphological staining.

Sensitivity of an ISH assay can be adapted by employing various degrees of hybridization stringency. As the hybridization conditions become more stringent, a greater degree of complementarity is required between the probe and target to form and maintain a stable duplex. Stringency is increased by adapting hybridization conditions e.g. by raising assay temperature or lowering salt concentration of the hybridization solution. After hybridization, slides are washed in a solution generally containing reagents similar to those found in the hybridization solution with washing time varying from minutes to hours depending on required stringency. (see e. g. “Darby, Ian A., and Tim D. Hewitson. 2006. In situ hybridization protocols. Totowa, N.J.: Humana Press; or Schwarzacher, Trude, and J. Heslop-Harrison. 2000. Practical in situ hybridization. Oxford, UK: BIOS; or Buzdin, Anton, and Sergey Lukyanov. 2007. Nucleic acids hybridization modern applications. Dordrecht: Springer).

Probes used in the ISH analysis may be either RNA or DNA oligonucleotides or polynucleotides and may contain not only naturally occurring nucleotides but their analogs like digoxygenin labeled dCTP, or biotin labeled derivatives e.g. biotin dcTP 7-azaguanosine.

Probes should have sufficient complementarity to the target nucleic acid sequence of interest so that stable and specific binding occurs between the target nucleic acid sequence and the probe. The degree of complementarity required for stable hybridization varies with the stringency of the hybridization and/or wash buffer. Preferably, probes with complete complementarity to the target sequence are used in the present invention. (see e. g., Sambrook, J., et al., Molecular Cloning A Laboratory Manual, Cold Spring Harbor Press, (1989))

A person skilled in the art will appreciate that the choice of probe depends on the characteristics of the target gene of interest. Probes may be genomic DNA, cDNA, or RNA cloned in a plasmid, phage, cosmid, YAC, Bacterial Artificial Chromosomes (BACs), viral vector, or any other suitable vector. Probes may be cloned or synthesized chemically by conventional methods (see, e. g., Sambrook, supra). In the present invention, probes are preferably labeled with a fluorophor. Examples of fluorophores include, but are not limited to, rare earth chelates (europium chelates), Texas Red, rhodamine, fluorescein, or dansyl. Multiple probes used in the assay may be labeled with more than one distinguishable fluorescent or indirect label.

After processing for ISH, the slides may be analyzed by standard techniques of microscopy. Briefly, each slide is observed using a microscope equipped with appropriate excitation filters, dichromic, and barrier filters. For FISH filters are chosen based on the excitation and emission spectra of the fluorophores used.

Typically, hundreds of cells are scanned in a tissue sample and quantification of the specific target nucleic acid sequence is determined in the form of fluorescent spots, which are counted relative to the number of cells. As provided herein, determination of FGFR1, FGFR2 and FGFR3 overexpression provides a much more effective indication of the likelihood that a pan FGFR inhibitor therapy will be effective, preferably a therapy with compounds of formula (I), (II), (III), (IV) and/or (V), most preferred with compounds of formula (I) all of them can be present in form of their salt, solvate and/or solvates of the salt.

The scoring is defined as follows:

| Staining score | Microscope objective scoring |
|---|---|
| 0 | No staining or less than 1 dot to every 10 cells |
| | (40X magnification) |
| 1 | 1-3 dots/cell (visible at 20-40X magnification) |
| 2 | 4-10 dots/cell. Very few dot clusters (visible |
| | at 20-40X magnification) |
| 3 | >10 dots/cell. Less than 10% positive cells have |
| | dot clusters (visible at 20X magnification) |
| 4 | >10 dots/cell. More than 10% positive cells have |
| | dot clusters (visible at 20X magnification) |

Eligible to treatment with a pan FGFR inhibitor according to the current invention are those showing a scoring of 3 of either one FGFR isoform (FGFR1, FGFR2 or FGFR3) or as a sum of all three FGFR isoforms preferred eligible are those where the tumor tissue samples show a score of at least 4, especially preferred are those having a score of more than 4.

Cancer according to the current invention is preferably head and neck cancer, especially preferred is squameous cell carcinoma of head and neck. Even more preferred is squameous cell carcinoma of head and neck wherein the sum of scoring is at least 6 and even more preferred wherein at least one of FGFR1, FGFR2 or FGFR3 has a scoring of at least 3.

In another embodiment the cancer is esophageal cancer, preferably showing as the sum of scoring at least 5. Even more preferred at least one of FGFR1, FGFR2 or FGFR3 has a scoring of at least 4.

Another preferred embodiment is ovarian cancer especially preferred when the sum of the scoring is at least 9.

In a further embodiment the cancer is lung cancer, preferably NSCLC, even more preferred squamous cell lung carcinoma. The sum of the scoring is preferably at least 5, even more preferred at least 7 and most preferred at least 9.

In another embodiment the cancer is colon cancer, preferably showing a sum of scoring of at least 4.

In another embodiment the cancer bladder cancer, preferably showing as a sum of the scoring at least 5.

In another embodiment the current invention is directed to a method of treatment of cancer in a subject by administering an effective amount of a pan FGFR inhibitor, wherein the subject is one for whom the sum of FGFR1, FGFR2 and/or FGFR3 mRNA in a tumor tissue sample from the subject has been found to be overexpressed

### EXAMPLES

### Material and Methods

### RNA Extraction and Quantitative Real-Time Polymerase Chain Reaction (RT-PCR)

Xenograft tumor pieces were immediately snap-frozen on dry ice and total RNA was extracted using Trizol method. Integrity of obtained RNA was checked on a Bioanalyzer (Agilent). For reverse transcription, 1 μg of total RNA was first digested with RNase-free DNase I for 15 min at room temperature and then reverse-transcribed using Promiscript in a total reaction volume of 40 μl according to the standard protocol of the kit supplier. After inactivation of the enzyme by heating for 15 min to 65° C., the obtained cDNA was diluted to a final volume of 150 μl with bidest. water and 4 μl were chosen per PCR reaction in a final volume reaction volume of 20 μl using TaqMan Universal Master Mix (2×) under standard cycler conditions (see TaqMan User Guide, Applied Biosytems for details) and the ABI PRISM 9600 sequence detection system. DNA sequences of PCR primers and FAM-labelled probes were designed by Primer Express 1.5 software (Applied Biosystems) and are summarized in Table1. Concentration of primers was 300 nM and of labelled probes 150 nM, respectively. Comparable amplification efficiencies for all primer/probe sets were checked by standard dilution curves. Expression was calculated using the ddCt method described by Livak and Schmittgen (Methods. 2001 December; 25(4):402-8.) with the exception that the normalized expression level of each FGFR mRNA was calculated using the formula: Expression=2(20−dCt), where dCt is the difference in Ct value between the gene of interest and the reference gene. Ct values were corrected for ribosomal protein L32, beta-2 microglobulin, cytosolic beta actin and glyceraldehyde 3-phosphate dehydrogenase mRNA levels to exclude different starting amounts of total RNA. We observed that the calculated expression level was independent of the housekeeping gene used. We therefore decided to normalize all FGFR mRNA expression data to L32 ribosomal protein. The resulting expression levels shown in Table 2 & 3 are the mean of three independent experiments (+/−) SD and are given in arbitrary units.

### Quantification of FGFR1 Gene Copy Number Gain.

Genomic DNA from xenograft tumors was isolated using DNeasy genomic DNA extraction kit from Qiagen. 0.5 ng geniomic DNA per samples was analysed for FGFR gene copy number gain using FGFR1 TaqMan Gene Copy Number Assay from Life Technologies. Results for FGFR were normalized to single copy reference gene RNase P as given in the protocol of the supplier. All xenograft models considered to be FGFR1 amplified according to table number 2 showed a higher signal intensity than expected for a single copy gene.

### In Vivo

### HN9897

Female, 6-8 weeks old immunocompromised nu/nu mice (19-27 g) from Harlan-Winkelmann (Germany) were used for the patient-derived HN9798 Head & Neck squamous cell carcinoma study.

Experiment was initiated after a minimal acclimatization period of 6 days. Mice were kept in a 12 hours light/dark cycle, food and water was available ad libitum and housing temperature was 20-26° C. Mice were randomly assigned to 2 experimental groups, ten mice per group. At the initiation of the treatment, animals were marked by ear-coding and the identification labels for each cage contained the following information: number of animals, sex, strain, receiving date, treatment, study number, group number, and the starting date of the treatment.

Tumor fragments from stock mice inoculated with selected primary human Head & Neck cancer tissues (HN9798) were harvested and used for inoculation onto female nu/nu nude mice. Each mouse was inoculated subcutaneously at the right flank with one tumor fragment (2×2 mm). The treatments were started on day 6 post implantation when mean tumor size reached approximately 0.075 cm³. Tumors were sampled when mice in the control group reached sacrificing criteria, and final tumor weights were measured on day 50 post inoculation.

Tumor size was measured twice weekly in two dimensions using a caliper, and the volume was expressed in mm³ using the formula: V=0.5a×b² where a and b are the long and short diameters of the tumor, respectively. The tumor size was then used for T/C values. T-C was calculated with T as the time (in days) required for the mean tumor size of the treatment group to reach a predetermined size (e.g. 1000 mm³), and C was the time (in days) for the mean tumor size of the control group to reach the same size. The T/C value was an indication of anti-tumor effectiveness; T and C were the mean volume of the treated and control groups, respectively, on a given day.

Descriptive statistics for all groups were performed on final tumor areas and tumor weights at day of the necropsy. Statistical analysis was assessed using the SigmaStat software. A one-way analysis of variance was performed, and differences to the control were compared by a multiple comparison using the Dunn's method.

### ES204

Female, 6-8 weeks old immunocompromised nu/nu mice (18-24 g) from Vital River (China) were used for the patient-derived ES204 esophagus squamous cell carcinoma study.

Experiment was initiated after a minimal acclimatization period of 6 days. Mice were kept in a 12 hours light/dark cycle, food and water was available ad libitum and housing temperature was 20-26° C. All the procedures related to animal handling, care, and the treatment in this study were performed according to the guidelines approved by the Institutional Animal Care and Use Committee (IACUC) of CrownBio following the guidance of the Association for Assessment and Accreditation of Laboratory Animal Care (AAALAC).

Mice were randomly assigned to four experimental groups, ten mice per group. At the initiation of the treatment, animals were marked by ear-coding and the identification labels for each cage contained the following information: number of animals, sex, strain, receiving date, treatment, study number, group number, and the starting date of the treatment.

Tumor fragments from stock mice inoculated with selected primary human esophagus cancer tissues (ES204) were harvested and used for inoculation onto female nu/nu nude mice. Each mouse was inoculated subcutaneously at the right flank with one tumor fragment (2-3 mm in diameter). The treatments were started on day 25 post implantation when mean tumor size reached approximately 100-150 mm³. Tumors were sampled when mice in the control group reached sacrificing criteria, and final tumor weights were measured on day 23 post treatment start.

Tumor size was measured twice weekly in two dimensions using a caliper, and the volume was expressed in mm³ using the formula: V=0.5a×b² where a and b are the long and short diameters of the tumor, respectively. The tumor size was then used for calculations of T/C values. T-C was calculated with T as the time (in days) required for the mean tumor size of the treatment group to reach a predetermined size (e.g. 1000 mm³), and C was the time (in days) for the mean tumor size of the control group to reach the same size. The T/C value was an indication of anti-tumor effectiveness; T and C were the mean volume of the treated and control groups, respectively, on a given day.

Descriptive statistics for all groups were performed on final tumor areas and tumor weights at day of the necropsy. Statistical analysis was assessed using the SigmaStat software. A one-way analysis of variance was performed, and differences to the control were compared by a multiple comparison using the Dunn's method.

### OVX1023

Female, 5-7 weeks old immunocompromised nu/nu mice (18-24 g) from Janvier (France) were used for the patient-derived OVX1023 ovarian carcinoma study.

The animals were housed in individually ventilated cages (TECNIPLAST Sealsafe™-IVC-System, TECNIPLAST, Hohenpeissenberg, Germany) and were kept under a 14L:10D artificial light cycle. The animals were monitored twice daily. Depending on group size, either type III cages or type II long cages were used. Dust-free bedding consisting of aspen wood chips with approximate dimensions of 5×5×1 mm (ABEDD®—LAB & VET Service GmbH, Vienna, Austria, Product Code: LTE E-001) were used. Additional nesting material was routinely added. The cages including the bedding and nesting material were changed weekly. The temperature inside the cages was maintained at 25±1° C. with a relative humidity of 45-65%. The air change (AC) rate in the cages was kept at 60 AC/h. All materials were autoclaved prior to use. Animals were fed Autoclavable Teklad Global 19% Protein Extruded Diet (T.2019S.12, Harlan Laboratories). All animals had access to sterile filtered and acidified (pH 2.5) tap water. Bottles were autoclaved prior to use and changed twice a week. Food and water were provided ad libitum. All materials were autoclaved prior to use.

All experiments were approved by the local authorities, and were conducted according to all applicable international, national and local laws and guidelines. Only animals with unobjectionable health were selected to enter testing procedures. Animals were routinely monitored twice daily on working days and daily on weekends and public holidays.

Mice were randomly assigned to four experimental groups, ten mice per group. Animals were arbitrarily numbered during tumor implantation or at the beginning of a dose finding experiment using radio frequency identification (RFID) transponders. Each cage was labeled with a record card indicating animal species, strain, source, gender, delivery date, experiment number, date of tumor implantation, date of randomization, tumor histotype, tumor number and passage, group identity, test compound, dosage, schedule, and route of administration.

Tumor fragments from stock mice inoculated with selected primary human esophagus cancer tissues (OVX1023) were harvested and used for inoculation onto female nu/nu nude mice. Each mouse was inoculated subcutaneously at the right flank with one tumor fragment (4-5 mm in diameter). Animals and tumor implants were monitored daily until the maximum number of implants showed clear signs of beginning solid tumor growth.

The treatments were started on day 49 post implantation when mean tumor size reached approximately 100-150 mm³. Tumors were sampled when mice in the control group reached sacrificing criteria, and final tumor weights were measured on day 50 post treatment start.

Tumor size was measured twice weekly in two dimensions using a caliper, and the volume was expressed in mm³ using the formula: V=0.5a×b² where a and b are the long and short diameters of the tumor, respectively. The tumor size was then used for calculations of T/C values. T-C was calculated with T as the time (in days) required for the mean tumor size of the treatment group to reach a predetermined size (e.g. 1000 mm³), and C was the time (in days) for the mean tumor size of the control group to reach the same size. The T/C value was an indication of anti-tumor effectiveness; T and C were the mean volume of the treated and control groups, respectively, on a given day.

Descriptive statistics for all groups were performed on final tumor areas and tumor weights at day of the necropsy. Statistical analysis was assessed using the SigmaStat software. A one-way analysis of variance was performed, and differences to the control were compared by a multiple comparison using the Dunn's method.

For all other tumors as listed in table 2 & 3 the experiments were carried out in an analogous way as described above.

TABLE 1
Sequences of RT-PCR primer/probes used for
mRNA quantification (all in 5′-3′) orientation.
Human Fibroblast Growth Factor Receptor-1:

| | |
|---|---|
| forward | GGCCCAGACAACCTGCCTTA |
| probe | CCACCGACAAAGAGATGGAGGTGCTT |
| reverse | TGCGTCCTCAAAGGAGACAT |

Human Fibroblast Growth Factor Receptor-2:

| | |
|---|---|
| forward | GCTGCTGAAGGAAGGACACA |
| probe | AGCCAGCCAACTGCACCAACGAA |
| reverse | GCATGCCAACAGTCCCTCA |

Human Fibroblast Growth Factor Receptor-3:

| | |
|---|---|
| forward | CTCGGGAGATGACGAAGAC |
| probe | CTGTGTCCACACCTGTGTCCTCA |
| reverse | CGGGCCGTGTCCAGTAA |

Human cytosolic beta-Actin

| | |
|---|---|
| forward | TCCACCTTCCAGCAGATGTG |
| probe | ATCAGCAAGCAGGAGTATGACGAGTCCG |
| reverse | CTAGAAGCATTTGCGGTGGAC |

Human beta-2 microglobulin:

| | |
|---|---|
| forward | GTCTCGCTCCGTGGCCTTA |
| probe | TGCTCGCGCTACTCTCTCTTTCTGGC |
| reverse | TGGAGTACGCTGGATAGCCTC |

Human L32 ribosomal protein:

| | |
|---|---|
| forward | CTGGTCCACAACGTCAAGGA |
| probe | TGGAAGTGCTGCTGATGTGCAA |
| reverse | AGCGATCTCGGCACAGTAAGA |

Human glyceraldehyde 3-phosphate dehydrogenase:

| | |
|---|---|
| forward | CTGGGCTACACTGAGCACCA |
| probe | TGGTCTCCTCTGACTTCAACAGCGACAC |
| reverse | CAGCGTCAAAGGTGGAGGAG |

TABLE 2
Correlation between FGFR-mRNA expression levels, FGFR1 copy
number gain and treatment efficacy to compounds of formula (I) in
vivo. Total RNA from xenograft tumors was isolated and quantified
for FGFR1 mRNA by Real Time PCR as described in Material &
Methods. In parallel, genomic DNA was isolated and FGFR1 gene
copy number gain was quantified using TaqMan copy number assay.
All models were considered to be gene-amplified in which the
FGFR1 signal intensity was stronger than for single-copy gene
(RNAse P) All models in which the tumor weight was reduced
upon treatment with compounds of formula (I) by at least
50% were considered to be efficacious in vivo.

| | Cancer | FGFR1 | FGFR1 | in vivo |
|---|---|---|---|---|
| Model | type | mRNA | amplified | efficacy |
| ES204 | esophageal | 72914 | no | yes |
| H520 | lung | 30407 | yes | no |
| LXFL1121 | lung | 18937 | yes | yes |
| LU1901 | lung | 16633 | yes | no |
| MFE280 | endometrial | 15018 | no | yes |
| OVXF1023 | ovarian | 12632 | no | yes |
| H1581 | lung | 11135 | yes | yes |
| H1703 | lung | 8667 | yes | no |
| Colo699 | lung | 8227 | yes | no |
| LXFE211 | lung | 7817 | no | yes |
| A2780 | ovarian | 5565 | no | yes |
| LXFA1584 | lung | 5442 | yes | no |
| DMS114 | lung | 5346 | yes | yes |

TABLE 3
Correlation between FGFR1, 2 and 3-mRNA expression level and treatment efficacy to
compounds of formula (I) in vivo. Tumor RNA was isolated and FGFR1-3 mRNA levels
were quantified by RT-PCR as described under Material & Methods. All models in
which the tumor weight was reduced upon treatment with compounds of formula (I) by
at least 50% were considered to be efficacious in vivo.

| | | FGFR1 | FGFR2 | FGFR3 | Total FGFR | In vivo |
|---|---|---|---|---|---|---|
| Model | Cancer type | mRNA | mRNA | mRNA | mRNA | efficacy |
| SNU16 | gastric | 83 | 89184 | 126 | 89393 | yes |
| H716 | colon | 12 | 78179 | 143 | 78335 | yes |
| ES204 | esophageal | 72914 | 2140 | 114 | 75167 | yes |
| HN9897 | head&neck | 277 | 960 | 51495 | 52732 | yes |
| RT112 | bladder | 99 | 1127 | 41267 | 42493 | yes |
| H520 | lung | 30407 | 9 | 401 | 30817 | no |
| H1581 | lung | 11135 | 12817 | 397 | 24348 | yes |
| MFM223 | breast | 1062 | 17770 | 24 | 18855 | yes |
| LXFL1121 | lung | 18937 | 28 | 134 | 19099 | yes |
| LU1901 | lung | 16633 | 1 | 2 | 16636 | no |
| MFE280 | endometrial | 15018 | 381 | 230 | 15629 | yes |
| OVXF1023 | ovarian | 12632 | 2346 | 224 | 15202 | yes |
| OMP2 | myeloma | 25 | 0 | 13066 | 13092 | no |
| HepG2 | liver | 704 | 916 | 6778 | 8398 | no |
| LU299 | lung | 3615 | 4742 | 1150 | 9507 | yes |
| LXFE211 | lung | 7817 | 11 | 474 | 8302 | yes |
| LU0697 | lung | 1884 | 980 | 6156 | 9020 | yes |
| H1703 | lung | 8667 | 68 | 187 | 8923 | no |
| Colo699 | lung | 8227 | 360 | 77 | 8664 | yes |
| HN366 | head&neck | 214 | 599 | 7297 | 8110 | yes |
| KYSE-140 | esophageal | 1520 | 4802 | 1366 | 7688 | no |
| LXFA1584 | lung | 5442 | 248 | 1627 | 7318 | no |
| HNXF908 | head&neck | 643 | 4311 | 1201 | 6155 | yes |
| DMS114 | lung | 5346 | 187 | 47 | 5580 | yes |
| A2780 | ovarian | 5565 | 0 | 13 | 5578 | yes |

| | T/C^{2.} at day 44 |
|---|---|
| HN9897 | post treatment! start |
| Vehicle ^{1.} 10 ml/kg 2QD po | 1 |
| Cpd of formula (I) 50 mg/kg 2QD po | 0.08 |

^{1.} 10% Ethanol. 40% Solutol HS15, 50% NaCl 0.9%
^{2.}T/C: Tumor with compounds of formula (I) treated mean/Tumor vehicle treated mean (Volume)

The same finding applied to a patient-derived esophageal squamous cell tumor with an extremely strong FGFR1-mRNA overexpression [ES204 in table 2] when tested for in vivo efficacy upon treatment with compounds of formula (I) in monotherapy:

| | T/C^{2.} at day 23 |
|---|---|
| ES204 | post treatment start |
| Vehicle ^{1.} 10 ml/kg 2QD po | 1 |
| Cpd of formula (I) 50 mg/kg 2QD po | 0.1 |

^{1.} 10% Ethanol. 40% Solutol HS15, 50% H₂O
^{2.}T/C: Tumor with compounds of formula (I) treated mean/Tumor vehicle treated mean (Volume)

A high FGFR1 mRNA overexpressing, patient-derived ovarian cancer model [OVXF1023 in table 2] showed also a very high in vivo efficacy:

| | T/C^{2.} at day 38 |
|---|---|
| OVXF1023 | post treatment start |
| Vehicle ^{1.} 10 ml/kg 2QD 5 days | 1 |
| on- 2 days off po | |
| Cpd of formula (I) 35 mg/kg 2QD 5 days | 0.2 |
| on- 2 days off po | |

^{1.}
^{2.}10% Ethanol. 40% Solutol HS15, 50% H₂O
^{3.} T/C: Tumor with compounds of formula (I) treated mean/Tumor vehicle treated mean (Volume)

To better quantify the amount of tumor FGFR1-3 RNA expression, we performed RNA in situ hybridization with selected models from table 3. Using FGFR1-, FGFR2- or FGFR3-specific probes, the RNA is stained in 5 μm slides of formalin-fixed, paraffin embedded xenograft tumors and quantified using a light microscope by a scoring system (see material & methods section for details).

TABLE 4
FGFR1-3 RNA in situ hybridization scoring of selected
xenograft tumor models using FFPE slides and probes
that are specific for either FGFR1, FGFR2 or FGFR3.

| | | FGFR1 | FGFR2 | FGFR3 | |
|---|---|---|---|---|---|
| | Cancer | RNA-ISH | RNA-ISH | RNA-ISH | In vivo |
| Model | type | score | score | score | efficacy |
| H716 | colon | 0 | 4 | 0 | yes |
| ES204 | esophageal | 4 | 1 | 0 | yes |
| HN9897 | head&neck | 1 | 1 | 4 | yes |
| RT112 | bladder | 0 | 1 | 4 | yes |
| H520 | lung | 4 | 1 | 2 | no |
| H1581 | lung | 4 | 4 | 1 | yes |
| LXFL1121 | lung | 4 | 0 | 1 | yes |
| LU1901 | lung | 4 | 1 | 2 | no |
| OVXF1023 | ovarian | 4 | 3 | 2 | yes |
| LU299 | lung | 4 | 3 | 3 | yes |
| LXFE211 | lung | 4 | 1 | 2 | yes |
| LU0697 | lung | 3 | 2 | 3 | yes |
| H1703 | lung | 4 | 1 | 1 | no |
| Colo699 | lung | 4 | 3 | 3 | yes |
| HN366 | head&neck | 2 | 3 | 4 | yes |
| LXFA1584 | lung | 4 | 2 | 3 | no |
| HNXF908 | head&neck | 1 | 2 | 3 | yes |
| DMS114 | lung | 4 | 1 | 1 | yes |

FGFR1, 2 and 3 mRNA expression levels in tumor tissue samples was quantified by RNA in situ hybridization using RNAscope technology from ACD (Advanced Cell Diagnostics, Inc., 3960 Point Eden Way, Hayward, Calif. 94545, USA) according to the manual of the supplier. FGFR1-3 probes are also commercially available from ACD:

### RNAscope® 2.0 HD Reagent Kit (RED) #310036,

### Probe—Hs-FGFR1 #310071

### Probe—Hs-FGFR2 #311171

### Probe—Hs-FGFR3 #310791

Scoring was performed according to the following:

| Staining score | Microscope objective scoring |
|---|---|
| 0 | No staining or less than 1 dot to every 10 cells |
| | (40X magnification) |
| 1 | 1-3 dots/cell (visible at 20-40X magnification) |
| 2 | 4-10 dots/cell. Very few dot clusters (visible |
| | at 20-40X magnification) |
| 3 | >10 dots/cell. Less than 10% positive cells have |
| | dot clusters (visible at 20X magnification) |
| 4 | >10 dots/cell. More than 10% positive cells have |
| | dot clusters (visible at 20X magnification) |

A lack of drug sensitivity despite high FGFR expression scoring can be explained by resistance mechanisms, e.g. LU1901 is a c-MET-overexpressing tumor and H520 is a has-mutated tumor—both mechanisms have been described to confer insensitivity to FGFR inhibitors.

TABLE 5
FGFR1-3 RNA in situ hybridization scoring data of selected patients from clinical trial
NCT01976741 using formalin-fixed, paraffin embedded (FFPE) slides and probes that are specific for
either FGFR1, FGFR2 or FGFR3. Patients were included into the trial if at least one of FGFR1, FGFR2
or FGFR3 has a scoring of at least 3. As shown in table 5, eight out of nine patients with such a scoring
result showed Stable Disease (SD) according to RECIST (Response Evaluation Criteria In Solid
Tumors-a set of published rules that define when cancer patients improve (“respond”), stay the same
(“stable”) or worsen (“progression”) during treatments-New response evaluation criteria in solid
tumors: Revised RECIST guideline (version 1.1) (European Journal of Cancer 45 (2009) 228-247) CT
scan evaluation criteria after 2 cyles (C2 = 6 weeks) or even after 5 cycles (C5 = 15 weeks) treatment
with a compound of formula (I) at 800 mg BID (“bis in die” = twice daily). Only one FGFR mRNA-
positive patient had progressive disease (PD) and was withdrawn from the trial.

| | | | | | RECIST | RECIST |
|---|---|---|---|---|---|---|
| | | | | | response | response |
| | | FGFR mRNA | | | after C2 | after C5 |
| | | RNA-ISH score | | | (% tumor | (% tumor |
| Pat ID | Tumor type | FGFR1 | FGFR2 | FGFR3 | change) | change) |
| 100010010 | sqNSCLC | 1 | 2 | 3 | SD (0) | SD (+8) |
| 580010016 | sqNSCLC | 1 | 3 | 4 | SD (−5) | SD (−6) |
| 580010012 | sqNSCLC | 2 | 3 | 3 | SD (−10) | SD (−10) |
| 580010022 | Bladder | 1 | 1 | 3 | SD (−3) | SD (−2) |
| 100020012 | sqNSCLC | 2 | 3 | 3 | SD (−10) | |
| 560020029 | sqNSCLC | 3 | 3 | 3 | SD (+4) | SD (+5) |
| 580010038 | Unknown primary | 0 | 3 | 4 | PD (+26) | |
| 560020064 | SCLC | 4 | 0 | 0 | SD (−2) | |
| 100010018 | Mesothelioma | 3 | 3 | 2 | SD (+14) | |

1. A pan FGFR inhibitor for use in the treatment of cancer in a subject, wherein the subject is one for whom the sum of FGFR1, FGFR2 and/or FGFR3 mRNA in a tumor tissue sample from the subject has been found to be overexpressed.

2. A pan FGFR inhibitor for use according to claim 1 wherein the mRNA overexpression is characterized by a scoring of at least 4 by in situ hybridization of the sum of FGFR1, FGFR2 and/or FGFR3 mRNA of a tumor tissue sample.

3. A pan FGFR inhibitor according to claim 1 wherein the cancer is squameous cell carcinoma of head and neck.

4. A pan FGFR inhibitor according to claim 3 wherein the sum of scoring is at least 6.

5. A pan FGFR inhibitor according to claim 4 wherein at least one of FGFR1, FGFR2 or FGFR3 has a scoring of at least 3.

6. A pan FGFR inhibitor according to claim 1 wherein the cancer is esophageal cancer.

7. A pan FGFR inhibitor according to claim 6 wherein the sum of scoring is at least 5.

8. A pan FGFR inhibitor according to claim 7 wherein at least one of FGFR1, FGFR2 or FGFR3 has a scoring of at least 4.

9. A pan FGFR inhibitor according to claim 8 wherein FGFR1 has a scoring of at least 4.

10. A pan FGFR inhibitor according to claim 1 wherein the cancer is ovarian cancer.

11. A pan FGFR inhibitor according to claim 10 wherein the sum of the scoring is at least 9.

12. A pan FGFR inhibitor according to claim 1 wherein the cancer is lung cancer.

13. A pan FGFR inhibitor according to claim 12 wherein the sum of the scoring is at least 5.

14. A pan FGFR inhibitor according to claim 1 wherein the cancer is colon cancer.

15. A pan FGFR inhibitor according to claim 1 wherein the cancer is bladder cancer.

16. A pan FGFR inhibitor according to claim 15 wherein the sum of the scoring is at least 5.

17. Use of a pan FGFR inhibitor according to claim 1 wherein the panFGFR inhibitor is selected from the group consisting of compounds of formula (I), (II), (III), (IV) and (V) 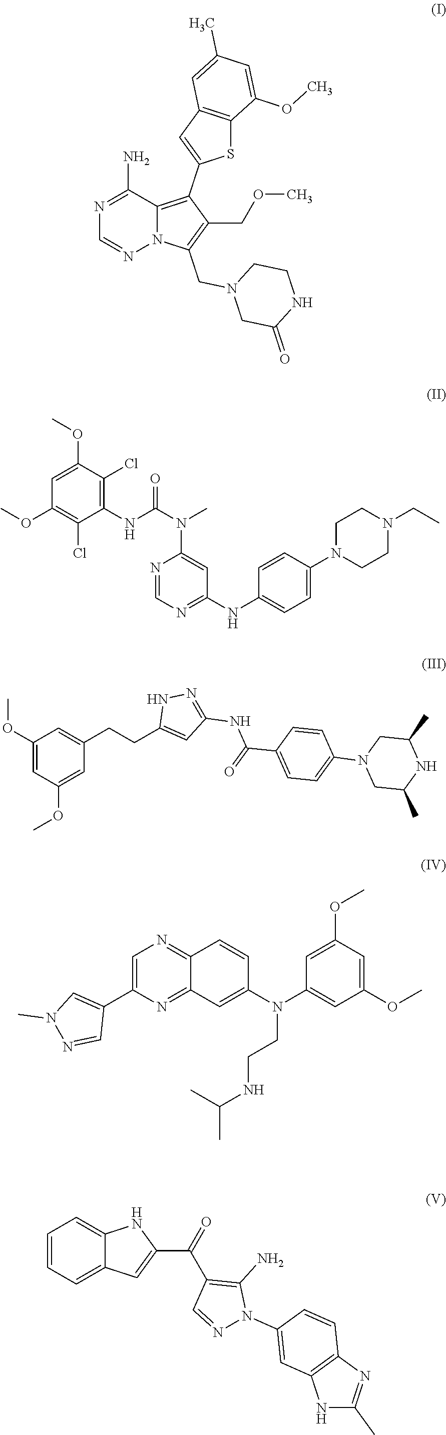
which can be present in form of their salts, solvates and/or solvates of the salts.

18. A method of identifying patients with cancer eligible for treatment with a pan FGFR inhibitor comprising testing a tumor tissue sample from the patient for the presence of FGFR1, FGFR2 and/or FGFR3 mRNA overexpression, wherein the patient is eligible for treatment with a pan FGFR inhibitor if the sum of the measured mRNA expression of FGFR1, FGFR2 and/or FGFR3 is overexpressed.

19. A method according to claim 18 wherein the cancer is squameous cell carcinoma of head and neck.

20. A method according to claim 18 wherein the cancer is esophageal cancer.

21. A method according to claim 18 wherein the cancer is ovarian cancer.

22. A method according to claim 18 wherein the cancer lung cancer.

23. A method according to claim 18 wherein the cancer colon cancer.

24. A method according to claim 18 wherein the cancer bladder cancer.

25. A method according to claim 18 wherein the pan FGFR inhibitor is a compound of formula I 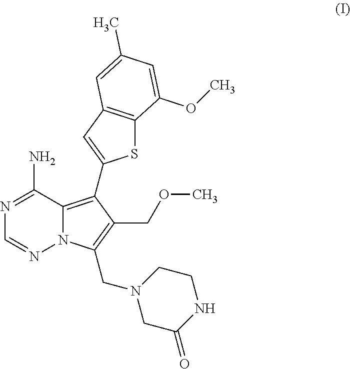 [SMILES: COCc1c(-c2cc3cc(C)cc(OC)c3s2)c2c(N)ncnn2c1CN1CCNC(=O)C1]
which can be present in form of its salt, solvate and/or solvates of the salt.

26. A method according to claim 18 wherein the pan FGFR inhibitor is a compound of formula (II) 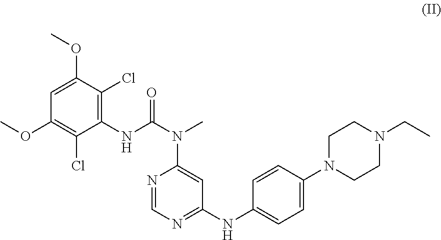 [SMILES: CCN1CCN(c2ccc(Nc3cc(N(C)C(=O)Nc4c(Cl)c(OC)cc(OC)c4Cl)ncn3)cc2)CC1]
which can be present in form of its salt, solvate and/or solvates of the salt.

27. A method according to claim 18 wherein the pan FGFR inhibitor is a compound of formula (III) 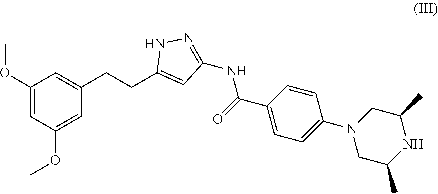 [SMILES: COc1cc(CCc2cc(NC(=O)c3ccc(N4C[C@@H](C)N[C@@H](C)C4)cc3)n[nH]2)cc(OC)c1]
which can be present in form of its salt, solvate and/or solvates of the salt.

28. A method according to claim 18 wherein the pan FGFR inhibitor is a compound of formula (IV) 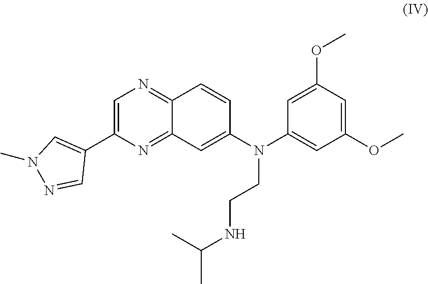
which can be present in form of its salt, solvate and/or solvates of the salt.

29. A method according to claim 18 wherein the pan FGFR inhibitor is a compound of formula (V) 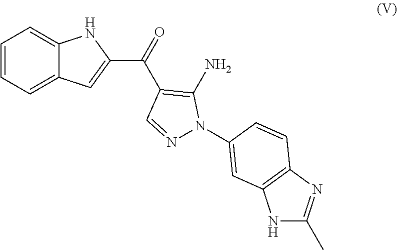 [SMILES: Cc1nc2ccc(-n3ncc(C(=O)c4cc5ccccc5[nH]4)c3N)cc2[nH]1]
which can be present in form of its salt, solvate and/or solvates of the salt.

30. A method according to claim 18 wherein patients showing a scoring of at least 4 by in situ hybridization of the sum of FGFR1, FGFR2 and/or FGFR3 of a formaldehyde-fixed cancer tissue sample are eligible for treatment with the respective inhibitor.

31. A method of treatment of cancer in a subject by administering an effective amount of a pan FGFR inhibitor, wherein the subject is one for whom the sum of FGFR1, FGFR2 and/or FGFR3 mRNA in a tumor tissue sample from the subject has been found to be overexpressed.
